(19) United States
(12) Patent Application Publication
(10) Pub. No.: US 2024/0268755 A1
(43) Pub. Date: Aug. 15, 2024

(54) SLEEP APNEA DETECTION USING WEARABLE DEVICE

(71) Applicant: SAMSUNG ELECTRONICS CO., LTD., Gyeonggi-Do (KR)

(72) Inventors: Chaoyi Kang, Porter Ranch, CA (US); Matthew Corbin Wiggins, San Jose, CA (US); DongHyun Lee, Seoul (KR); Amol Jayant Pangarkar, Fremont, CA (US)

(21) Appl. No.: 18/361,678

(22) Filed: Jul. 28, 2023

Related U.S. Application Data
(60) Provisional application No. 63/445,668, filed on Feb. 14, 2023.

(51) Int. Cl. A61B 5/00; A61B 5/145

(52) U.S. Cl. CPC A61B 5/4818; A61B 5/14542; A61B 5/7278; A61B 5/7282

(57) ABSTRACT

While a user is wearing a device during one or more sleep cycles, sensor signals are continuously monitored to detect a plurality of sleep sessions occurring during the one or more sleep cycles. Based on the sensor signals, amounts of valid SpO₂ data collected for the plurality of sleep sessions are measured. A merged sleep session is generated by combining selected sleep sessions of the plurality of sleep sessions occurring in a same sleep cycle based on a maximum amount of time between the selected sleep sessions and the amounts of valid SpO₂ data collected for the selected sleep sessions. An estimated apnea hypopnea index (eAHI) is estimated for the one or more sleep sessions including the merged sleep session. An indication of whether the user has sleep apnea is generated based on one or more of the eAHIs for the one or more sleep cycles.

### CROSS-REFERENCE TO RELATED APPLICATIONS

This application claims the benefit of U.S. Provisional Patent Application No. 63/445,668 filed on Feb. 14, 2023, which is fully incorporated herein by reference.

### TECHNICAL FIELD

This disclosure relates to detecting sleep apnea and, more particularly, to detecting sleep apnea using a wearable device.

### BACKGROUND

Sleep Apnea Hypopnea Syndrome is a condition in which a person experiences an abnormal pause or rate reduction in breathing during sleep. A hypopnea refers to a period of shallow breathing in a person during sleep. An apnea refers to a complete pause in a person's breathing during sleep. Apneic and hypopneic events disrupt sleep and lead to lower blood oxygen levels, which may contribute to long-term health complications. The Apnea-Hypopnea Index (AHI) is a diagnostic tool for determining the presence and severity of obstructive sleep conditions such as sleep apnea. AHI represents the average number of apneas and hypopneas a person experiences each hour during sleep. AHI may be determined by dividing the total number of apneic and hypopneic events by the total number of hours one sleeps. In general, to register as an event, an apnea or hypopnea must last at least 10 seconds or longer.

Sleep apnea is widely believed to be an under-detected condition in the population. This is due, in large part, to the way in which sleep apnea is detected. Detection of sleep apnea requires access to both specialized personnel and specialized sleep monitoring equipment. In the usual case, a subject that suspects an affliction with sleep apnea first consults a primary care physician to obtain a prescription or referral to a sleep specialist. The sleep specialist then coordinates the participation of the person in polysomnography (PSG), also known as a sleep study. PSG is the standard clinical test for detecting sleep disorders including sleep apnea. PSG utilizes highly specialized equipment that records the subject's brain waves, the oxygen level in the subject's blood, the subject's heart rate, and the subject's breathing during sleep. Eye and leg movements of the subject are also recorded. PSG is capable of measuring AHI based on a number of nasal pressure drops of at least a minimum amplitude and duration.

PSG may be performed at a clinic which requires that the subject sleep at the clinic in a highly monitored environment. Alternatively, PSG may be performed in the subject's home. Even when performed in the subject's home, PSG requires the use of highly specialized equipment. Both sleep study options are costly and require a sleep study specialist to evaluate the compiled over-night data to derive the AHI. There is a lack of direct-to-consumer, or over-the-counter, devices capable of accurately and reliably detecting sleep apnea.

### SUMMARY

In one or more embodiments, a method is performed within and by a wearable device including a hardware processor. The method includes, while a user is wearing the wearable device during one or more sleep cycles, continuously monitoring, by the hardware processor, sensor signals generated by sensors of the wearable device to detect a plurality of sleep sessions occurring during the one or more sleep cycles. The method includes measuring, by the hardware processor and based on the sensor signals, amounts of valid SpO₂ data collected for the plurality of sleep sessions. The method includes generating, by the hardware processor, a merged sleep session by combining selected sleep sessions of the plurality of sleep sessions occurring in a same sleep cycle based on a maximum amount of time between the selected sleep sessions and the amounts of valid SpO₂ data collected for the selected sleep sessions. The method includes estimating, by the hardware processor, an estimated apnea hypopnea index (eAHI) for the one or more sleep sessions including the merged sleep session. The method includes generating, by the hardware processor, an indication of whether the user has sleep apnea based on one or more of the eAHIs for the one or more sleep cycles.

The foregoing and other implementations can each optionally include one or more of the following features, alone or in combination. Some example implementations include all the following features in combination.

In some aspects, generating the indication includes, for each of the one or more sleep cycles, comparing the eAHI associated with the sleep cycle with a threshold eAHI and indicating that the user has sleep apnea in response to determining that the eAHI associated with at least one sleep cycle exceeds the threshold eAHI.

In some aspects, generating the merged sleep session includes determining that an insufficient amount of valid SpO₂ data was collected during a first sleep session of the plurality of sleep sessions of a selected sleep cycle and determining that a second sleep session of the plurality of sleep sessions occurs within a threshold amount of time after an end of the first sleep session in the selected sleep cycle. In response to determining that a sufficient amount of SpO₂ data is collected for the second sleep session, the eAHI associated with the selected sleep cycle is determined based on the SpO₂ data collected for the second sleep session.

In some aspects, the merged sleep session is formed of a first sleep session and a second sleep session. Estimating the eAHI includes, for the merged sleep session determining a first eAHI associated with the first sleep session based on a number of apneas during the first sleep session and determining a second eAHI associated with the second sleep session based on a number of apneas during the second sleep session. An eAHI for the merged sleep session is generated by combining the first eAHI and the second eAHI.

In some aspects, the method includes combining the first eAHI and the second eAHI using a weighted sum. The method includes determining the weighted sum using weights determined based on a duration of the first sleep session and a duration of the second sleep session.

In some aspects, the method includes varying the maximum amount of time between the selected sleep sessions when determining whether to merge the selected sleep sessions based on the amounts of valid SpO₂ data collected for a first sleep session of the selected sleep sessions considered for merging.

In some aspects, the maximum amount of time between the selected sleep sessions is increased in response to determining that the amount of valid SpO₂ data collected for the first sleep session is insufficient.

In one or more embodiments a system includes, a plurality of sensors configured to generate sensor signals. The system includes a hardware processor coupled to the plurality of sensors. The hardware processor is configured to continuously monitor the sensor signals while worn by a user during one or more sleep cycles. The hardware processor includes a sleep session detector configured to detect a plurality of sleep sessions occurring during the one or more sleep cycles based on the sensor signals. The hardware processor includes an SpO₂ quantifier configured to determine amounts of SpO₂ data collected for the plurality of sleep sessions based on the sensor signals. The hardware processor includes a sleep session manager configured to generate a merged sleep session by combining selected sleep sessions of the plurality of sleep sessions occurring in a same sleep cycle based on a maximum amount of time between the selected sleep sessions and the amounts of SpO₂ data collected for the selected sleep sessions. The hardware processor includes an apnea hypopnea index (AHI) estimator configured to estimate an estimated apnea hypopnea index (eAHI) for one or more of the plurality of sleep sessions including the merged sleep session. The hardware processor includes a sleep apnea detector configured to indicate, via a user interface of the system, whether the user has sleep apnea based on one or more eAHIs for the one or more sleep cycles.

The foregoing and other implementations can each optionally include one or more of the following features, alone or in combination. Some example implementations include all the following features in combination.

In some aspects, the sleep apnea detector is configured to, for each of the one or more sleep cycles, compare the eAHI associated with the sleep cycle with a threshold eAHI. The sleep apnea detector also is configured to indicate that the user has sleep apnea in response to determining that the eAHI associated with at least one sleep cycle exceeds the threshold eAHI.

In some aspects, the sleep session manager is configured to determine that an insufficient amount of valid SpO₂ data was collected during a first sleep session of the plurality of sleep sessions of a selected sleep cycle and determine that a second sleep session of the plurality of sleep sessions occurs within a threshold amount of time after an end of the first sleep session in the selected sleep cycle. The sleep session manager also is configured to, in response to determining that a sufficient amount of SpO₂ data is collected for the second sleep session, determine the eAHI associated with the selected sleep cycle based on the SpO₂ data collected for the second sleep session.

In some aspects, the merged sleep session is formed of a first sleep session and a second sleep session. For the merged sleep session, the AHI estimator is configure to determine a first eAHI associated with the first sleep session based on a number of apneas during the first sleep session and determine a second eAHI associated with the second sleep session based on a number of apneas during the second sleep session. The AHI estimator is configured to generate an eAHI for the merged sleep session by combining the first eAHI and the second eAHI.

In some aspects, the AHI estimator is configured to combine the first eAHI and the second eAHI using a weighted sum and determine the weighted sum using weights determined based on a duration of the first sleep session and a duration of the second sleep session.

In some aspects, the sleep session manager is configured to vary the maximum amount of time between the selected sleep sessions when determining whether to merge the selected sleep sessions based on the amounts of valid SpO₂ data collected for a first sleep session of the selected sleep sessions considered for merging.

In some aspects, the maximum amount of time between the selected sleep sessions is increased in response to determining that the amount of valid SpO₂ data collected for the first sleep session is insufficient.

In some aspects, the system may be implemented as a wearable device. The system may be an over-the-counter device.

In one or more embodiments, a computer program product includes a computer readable storage medium having program code stored thereon. The program code is executable by a hardware processor to perform the various operations described within this disclosure.

This Summary section is provided merely to introduce certain concepts and not to identify any key or essential features of the claimed subject matter. Many other features and embodiments of the invention will be apparent from the accompanying drawings and from the following detailed description.

### BRIEF DESCRIPTION OF THE DRAWINGS

The accompanying drawings show one or more embodiments; however, the accompanying drawings should not be taken to limit the invention to only the embodiments shown. Various aspects and advantages will become apparent upon review of the following detailed description and upon reference to the drawings.

FIG. 1 illustrates an example of a device for use with one or more embodiments described within this disclosure.

FIG. 2 illustrates an example implementation of Sleep Apnea Detection System (SADS) as executed by the device of FIG. 1.

FIG. 3 illustrates an example method of detecting sleep apnea.

FIG. 4 illustrates an example method of generating and merging sleep sessions in accordance with the inventive arrangements disclosed herein.

FIG. 5 illustrates an example method of determining whether the user has experienced moderate to severe sleep apnea as performed by the device of FIG. 1 executing the SADS.

### DETAILED DESCRIPTION

While the disclosure concludes with claims defining novel features, it is believed that the various features described herein will be better understood from a consideration of the description in conjunction with the drawings. The process(es), machine(s), manufacture(s) and any variations thereof described within this disclosure are provided for purposes of illustration. Any specific structural and functional details described are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to detecting sleep apnea and, more particularly, to detecting sleep apnea with a wearable device. In accordance with the inventive arrangements described within this disclosure, methods, systems, and computer-program products are provided that are capable of detecting sleep apnea in a human being using a readily available device. The device may be a wearable device that is widely available to a user as an over-the-counter (OTC) device.

In one or more example implementations, the inventive arrangements are capable of monitoring a user to determine time periods in which the user is asleep. The time period during which a user is considered to be sleeping is referred to as a sleep session. In one or more examples, based on defined criteria, different sleep sessions occurring in the same sleep cycle may be combined or merged into a single larger sleep session to acquire sufficient data for estimating AHI. The inventive arrangements are capable of determining an estimate of AHI, referred to herein as “eAHI,” for each of one or more sleep cycles based on the sleep sessions detected.

As discussed, in performing polysomnography (PSG), a sleep specialist calculates AHI for a subject based on a review of the clinical PSG data collected using highly specialized equipment. In PSG, AHI is calculated based on a number of nasal pressure drops in the user of at least a minimum amplitude and duration during sleep based on the clinical PSG data. In general because of the accuracy of the specialized equipment involved, AHI may be determined through observation of the user using PSG for a single sleep cycle.

Clinical PSG data, however, is not available or obtainable by an OTC (e.g., non-prescription) device. An OTC device lacks the complexity and sensors of specialized PSG equipment. In one or more example implementations, an OTC device configured as described herein is capable of using one or more alternative data sources to detect sleep apnea in a user. The inventive arrangements are capable of continuously monitoring or sensing oxygen saturation in the user's blood (e.g., Peripheral Oxygen Saturation or “SpO₂”) as an alternative data source over one or more sleep sessions occurring over one or more sleep cycles. Based on the SpO₂ levels determined, the OTC device is capable of generating an eAHI for each of one or more sleep cycles. The eAHI is based on SpO₂ data unlike the AHI.

In accordance with the inventive arrangements described herein, the device is capable of determining eAHI for each of one or more sleep cycles. Based on the determined eAHI value(s) of the sleep cycle(s), the device implements an automated pre-screening process for sleep apnea for a user. In some examples, the device may perform a pre-screening process based on the eAHI from a single sleep cycle. In other examples, given the use of SpO₂ as an alternative data source and the need for accuracy, the device may perform the pre-screening process based on the eAHI from each of a plurality of sleep cycles that occur within a predefined window of time.

Further aspects of the inventive arrangements are described below in greater detail with reference to the figures. For purposes of simplicity and clarity of illustration, elements shown in the figures are not necessarily drawn to scale. For example, the dimensions of some of the elements may be exaggerated relative to other elements for clarity. Further, where considered appropriate, reference numbers are repeated among the figures to indicate corresponding, analogous, or like features.

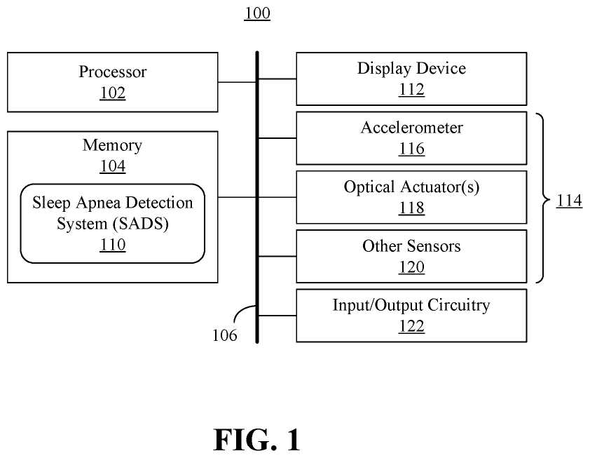

FIG. 1 illustrates an example of a device 100 for use with one or more embodiments described within this disclosure. Device 100 may be embodied or implemented as any of a variety of readily available consumer electronic devices. Device 100 may be considered an OTC device in that no prescription is required to obtain the device or use the device in accordance with the inventive arrangements described within this disclosure. In one or more example implementations, device 100 is implemented as a portable device such as, for example, a wearable device. As an illustrative and non-limiting example, device 100 may be implemented as a smart-watch, as a smart-ring, or other wearable accessory. The particular form factor of device 100 is not intended as a limitation so long as device 100 is able to perform the various functions/operations described herein in connection with sleep monitoring.

In the example, device 100 includes a processor 102, a memory 104, and interface circuitry 106 that couples various system components including memory 104 to processor 102. Processor 102 may be implemented as one or more processors. For example, processor 102 may be implemented as one or more circuits (e.g., as hardware) capable of carrying out instructions contained in program code. In an example, processor 102 is implemented as a central processing unit (CPU). Processor 102 may be implemented using a complex instruction set computer architecture (CISC), a reduced instruction set computer architecture (RISC), or other known architectures including embedded and/or mobile processor architectures. Example processors include, but are not limited to, processors having an x86 type of architecture (IA-32, IA-64, etc.), Power Architecture, ARM processors, and the like.

Memory 104 may include computer system readable media (e.g., physical memory devices). Such media may include computer-readable volatile and non-volatile media and computer-readable removable and non-removable media. For example, memory 104 can include computer-readable media in the form of volatile memory, such as random-access memory (RAM) and/or cache memory as used during execution of program code. Memory 104 also includes non-volatile memory. Examples of non-volatile memory may include a solid-state drive (SSD), flash memory, a read-only memory (ROM), an erasable programmable read-only memory (EPROM), electrically erasable programmable read-only memory (EEPROM), or other suitable memory.

Memory 104 stores computer readable instructions (also referred to as “program code”). Processor 102 is capable of performing the various operations described herein through execution of the computer readable instructions as accessed from memory 104. In one aspect, a Sleep Apnea Detection System (SADS) 110 is implemented in program code that is stored in memory 104 and is executable by processor 102. SADS 110 may be executed with an operating system also stored in memory 104 or without an operating system depending on the implementation of device 100. It should be appreciated that any data (e.g., sensor data derived from sensor generated signals) and/or program code used, generated, and/or operated upon by device 100 (e.g., processor 102) are functional data structures that impart functionality when employed as part of device 100.

Interface circuitry 106 is connective circuitry and may be implemented as, for example, an input/output (I/O) subsystem, interconnect circuitry, a communication bus, a memory interface, or a combination of one or more or all of the aforementioned examples.

Device 100 optionally includes a display device 112. Display device 112 is coupled to interface circuitry 106. Through operation of processor 102, display device 112 may generate a user interface and/or graphical user interface through which indications, notifications, messages, or other information may be communicated to a user. Display device 112 may be touch sensitive and capable of receiving touch input from a user. A touch sensitive display is capable of detecting contact, movement, gestures, and breaks in contact using any of a variety of available touch sensitivity technologies. Example touch sensitive technologies include, but are not limited to, capacitive, resistive, infrared, and surface acoustic wave technologies, and other proximity sensor arrays or other elements for determining one or more points of contact with a touch sensitive display and/or device.

In the example, device 100 includes a plurality of sensors 114 coupled to interface circuitry 106. Sensors 114 include an accelerometer 116. Accelerometer 116 is capable of generating accelerometer signals that indicate change of speed and direction of movement of device 100 in 3-dimensions. Sensors 114 also include one or more optical actuators 118. In one aspect, an example of an optical actuator is a pulse oximeter. Optical actuator(s) 118 are capable of measuring SpO₂ in a user. In one or more example implementations, an optical actuator is implemented as a photoplethysmogram (PPG) sensor or array. Optical actuator(s) 118 are capable of illuminating the skin of the user and generating signals (e.g., PPG signals) correlated with changes in light absorption that indicate volumetric variations in blood circulation.

Device 100 optionally includes one or more other sensors 120. Other sensors 120 may be coupled to interface circuitry 106. Examples of such other sensors may include, a camera, a microphone, a motion sensor, a light sensor, and a proximity sensor, a location sensor (e.g., a GPS receiver and/or processor) capable of providing geo-positioning sensor data, an electronic magnetometer (e.g., an integrated circuit chip) capable of providing sensor data that can be used to determine the direction of magnetic North for purposes of directional navigation, and an altimeter (e.g., an integrated circuit) capable of providing data indicating altitude.

Device 100 may include I/O circuitry 122. I/O circuitry 122 may be coupled to interface circuitry 106. I/O circuitry 122 implements one or more communication ports and/or wireless adapters. For example, I/O circuitry 122 may implement one or more wired communication ports and/or one or more wireless adapters over which data may be sent and/or received. Via I/O circuitry 122, device 100 may communicate with one or more other external devices and/or systems.

Device 100 is provided for purposes of illustration and not limitation. A device and/or system configured to perform the operations described herein may have a different architecture than illustrated in FIG. 1. The architecture may be a simplified version of the architecture described in connection with FIG. 1 that includes fewer components than shown. Alternatively, the architecture may be more complex and include additional components not illustrated in FIG. 1. Device 100 is an example of a data processing system or computer hardware. As noted, device 100 may be embodied in a wearable form factor.

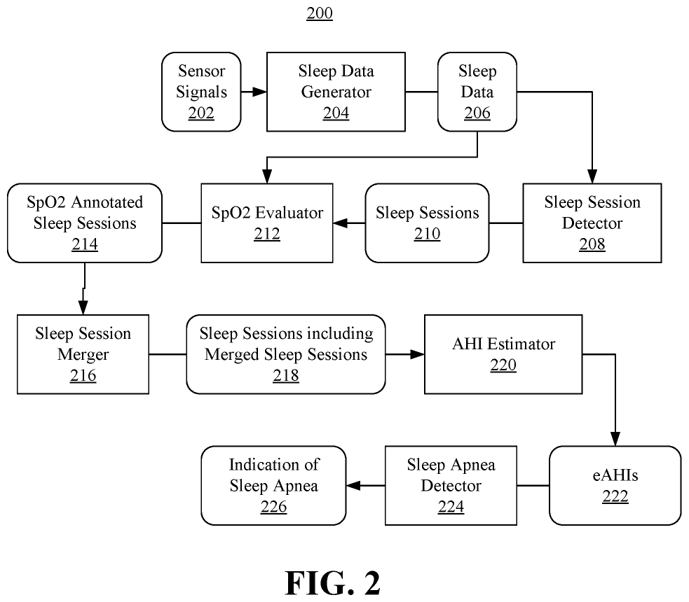

FIG. 2 illustrates an example architecture 200 that may be used to implement SADS 110 of FIG. 1. The various blocks described in connection with FIG. 2 may be implemented as program code and, as such, architecture 200 is executable by processor 102. FIG. 2 also illustrates an example data flow for architecture 200. The operations illustrated in FIG. 2 may be performed by device 100 while worn by a user through the course of one or more sleep cycles. Within this disclosure, the term “sleep cycle” refers to a period of time during which a user typically sleeps or attempts to sleep. A sleep cycle may refer to a night (e.g., a time in the evening extending until a time in the next morning), an afternoon, or the like depending on the particular schedule and/or sleep habits of the user. For purposes of illustration, a sleep cycle may refer to a period of time of approximately 8 hours during which a user typically sleeps.

In the example, sensors 114, e.g., accelerometer 116 and optical actuator(s) 118, generate sensor signals 202. Other sensors also may contribute to sensor signals 202. Accordingly, sensor signals 202 include accelerometer signals and PPG signals and may include additional sensor generated signals. While worn by the user, device 100 may continually generate sensor signals 202 and monitor sensor signals 202 as described herein. In general, SpO₂ data, as part of sleep data 206, may be generated at a sample rate of approximately 1 Hz or a greater sampling rate. Device 100 may monitor and/or analyze the sensor signals 202 in real time.

Sleep data generator 204 is capable of generating a time series of sleep data 206, e.g., time-stamped, from sensor signals 202. Sleep data 206 may include accelerometer data derived from the accelerometer signals, PPG data derived from PPG signals, SpO₂ data derived from PPG signals and/or data, heart rate data derived from PPG data, heart rate variability data, breathing data derived from accelerometer data and/or microphone detected data (e.g., audio), and the like.

Sleep session detector 208 receives sleep data 206 and is capable of detecting periods in which the user is considered to be asleep. The periods in which the user is considered to be asleep are referred to herein as “sleep sessions.” In one or more examples, sleep session detector 208 is capable of determining whether the user is asleep based on different types of sleep data 206 that may include one or more or all of accelerometer data, user behavior data, and physiological data of the user.

In one or more examples, sleep session detector 208 is capable of analyzing the accelerometer data from sleep data 206 to determine sleep sessions 210 based on detecting periods during which movement of the user is below a particular threshold of movement indicating the user is asleep. Accelerometer data may be used by sleep session detector 208 to determine information such as body position and arousal index to determine the start and/or end of a sleep session.

In one or more examples, sleep detector 208 is capable of analyzing audio data that may be included in sleep data 206. The audio data may be derived from microphone generated signals (e.g., audio signals) to detect noise from user movement, breathing, etc., to determine whether the user is asleep. For example, sounds such as user movement, breathing, etc. having a volume below a threshold or lack of detected speech may indicate that the user is sleeping. Conversely, sounds above the threshold and/or detected user speech may indicate that the user is not sleeping.

In one or more examples, sleep session detector 208 is capable of evaluating PPG amplitude from the PPG data, heart rate and/or heart rate variability, and/or breathing patterns to determine whether the user is asleep. Heart rate and heart rate variability, for example, tend to become constant or regulated while the user sleeps. In one or more examples, user behavior data such as historical sleep patterns of the user may be used to determine whether the user is asleep.

The examples described above in connection with detecting sleep sessions are provided for purposes of illustration only. It should be appreciated that detecting sleep sessions of the user may be performed by sleep session detector 208 using any of a variety of known techniques. Further, sleep session detector 208 may rely on any combination of the different types of sleep data 206 described herein, e.g., using a weighted sum and/or combination of the aforementioned factors against a predefined threshold score.

Sleep session detector 208 is capable of continuing to monitor sleep data 206 from sensor signals 202 to determine a start time that the user is believed to have fallen asleep (e.g., sleep onset) and continue to monitor to determine whether the user remains asleep. In response to determining that the user experiences an arousal from sleep or awakens (e.g., a sleep offset), sleep session detector 208 is capable of generating a sleep session that is correlated with, or includes, SpO₂ data collected from sleep onset on through sleep offset. Sleep session detector 208 is capable of outputting sleep sessions 210 and do so as detected where each sleep session has a defined start time corresponding to the sleep onset and a defined end time corresponding to the sleep offset.

In the example, selected portions of sleep data 206, e.g., SpO₂ data, is provided to SpO₂ evaluator 212. SpO₂ evaluator 212 is capable of evaluating the SpO₂ data in connection with sleep sessions 210. For example, SpO2 evaluator 212 is capable of determining whether the SpO₂ data for each sleep session is valid. Valid SpO₂ data, as described herein, is required to produce an accurate and reliable eAHI.

SpO₂ evaluator 212 determines whether SpO₂ data is valid based on several different criteria. SpO₂ evaluator 212 determines whether, for a given sleep session, the SpO₂ values are between 70% and 100%. In general, SpO₂ values less than 70% are considered unreliable and may be indicative of a measurement problem with respect to the optical actuator(s). SpO₂ evaluator 212 also determines whether a minimum number of SpO₂ values (e.g., samples) exist that are in the valid range (e.g., between 70% and 100%). As an illustrative and non-limiting example, a minimum of 3,600 SpO₂ values may be required, where each has a value between 70% and 100%. In addition, SpO₂ evaluator 212 determines whether the valid SpO₂ values, e.g., those between 70% and 100%, for a given sleep session cover at least 70% of the duration of the sleep session. If all of the foregoing criteria are met, the SpO₂ data for the sleep session is considered valid.

In one or more examples, SpO₂ evaluator 212 is capable of annotating each sleep session with an indicator that specifies whether the SpO₂ data for the sleep session is considered valid. In the example SpO₂ evaluator 212 is capable of outputting SpO₂ annotated sleep sessions 214. The SpO₂ annotated sleep sessions 214 may be provided to sleep session manager 216. A sleep session with valid SpO₂ data includes SpO₂ data covering 70% or more of the duration of the sleep session and, of that SpO₂ data, the values are between 70% and 100%.

Sleep session manager 216 is capable of merging selected ones of the sleep sessions based on criteria that includes whether the sleep sessions have valid SpO₂ data and a maximum amount of time between sleep sessions occurring in a same sleep cycle (e.g., during the same night). Further, the maximum amount of time that occurs between sleep sessions considered for combining into a merged sleep session may be varied based on the validity of the SpO₂ data for the respective sleep sessions being considered. Sleep session manager 216 outputs the resulting sleep sessions 218, which include any merged sleep sessions.

AHI estimator 220 evaluates sleep sessions 218 as output from sleep session manager 216 and generates eAHIs 222. AHI estimator 220 may generate an eAHI, e.g., one eAHI, for each sleep cycle. AHI estimator 220 may determine an eAHI based on a number of apnea events occurring in a given sleep session (e.g., including a merged sleep session). In one aspect, each apnea event may be detected as a drop in SpO₂ of a minimum amount or percentage that continues for a minimum amount of time. The eAHI, as determined by AHI estimator 220, also may depend on the total duration of apnea events with respect to the duration of the sleep session (or merged sleep session) in which the apnea events are detected.

In one or more embodiments, AHI estimator 220 generates an eAHI as a value calculated based on a number of SpO₂ dips occurring for a sleep cycle, a total duration of the sleep sessions of the sleep cycle, and/or a total duration of the apnea events (e.g., duration of SpO₂ dips of a predetermined magnitude or amount).

In one or more example embodiments, AHI estimator 220 may be implemented as a machine learning model that is trained with annotated or labeled data. Given sleep sessions and accompanying SpO₂ data, AHI estimator 220 may be trained to output an eAHI value that approximates an actual AHI value given clinical PSG data collected concurrently with the sleep session and accompanying SpO₂ data. The machine learning model may be trained to classify apnea hypopnea events based on factors such as number of SpO₂ reductions, reduction frequency, and reduction amplitude.

Sleep apnea detector 224 receives eAHIs 222 as generated for one or more sleep cycles. Sleep apnea detector 224 is capable of comparing the eAHI of each of one or more sleep cycles with a threshold. Based on the number of sleep cycles in which the eAHI exceeds the threshold, sleep apnea detector 224 outputs indication of sleep apnea 226 via a user interface of device 100. In another example, indication of sleep apnea 226 may be output from a communication port or a wireless adapter of I/O circuitry 122.

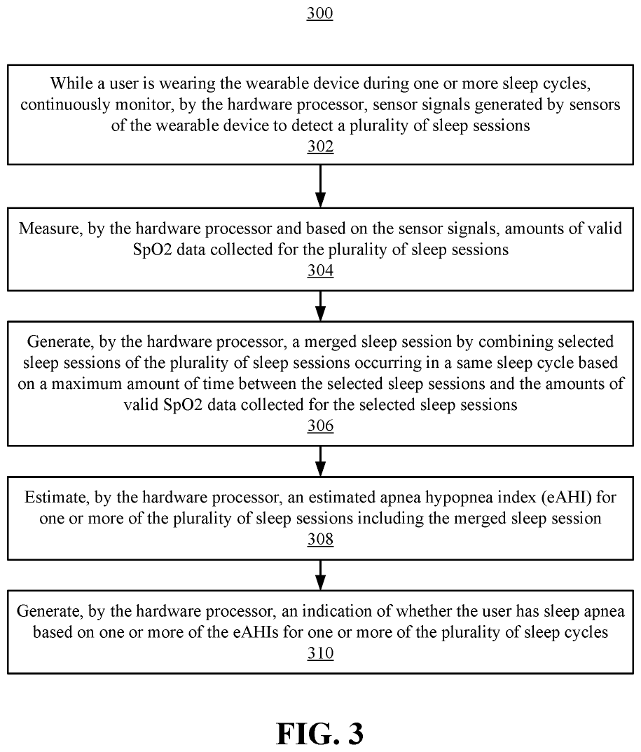

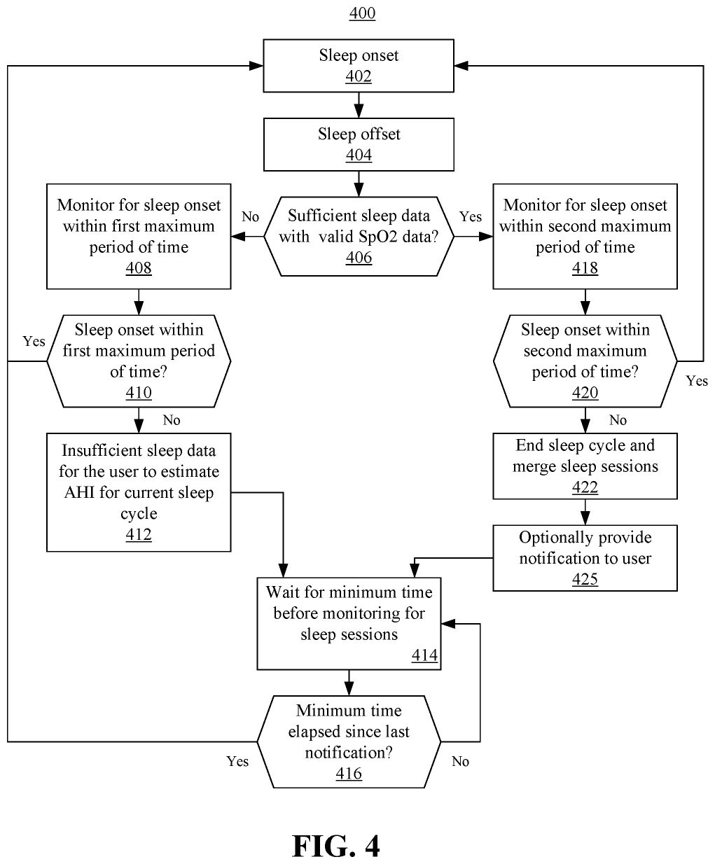

The architecture 200 of FIG. 2 is provided for purposes of illustration and not limitation. Architecture 200 may include other logic blocks (e.g., program code and/or circuitry) that interact with the blocks illustrated in FIG. 2 to effectuate certain control and/or decision-making functions as described in greater detail in connection with FIGS. 3, 4, and/or 5.

FIG. 3 illustrates an example method 300 of detecting whether a user has sleep apnea. Method 300 is performed by device 100 executing SADS 110. Method 300 may be performed over one or more sleep cycles.

In block 302, while a user is wearing device 100 during one or more sleep cycles, processor 102, continuously monitors sensor signals 202 generated by sensors 114 to detect a plurality of sleep sessions 210. In block 304, processor 102 in executing SpO₂ evaluator 212, measures amounts of valid SpO₂ data collected for the plurality of sleep sessions 210 based on sensor signals 202 (e.g., as converted into sleep data 206).

In block 306, processor 102 in executing sleep session manager 216, generates one or more merged sleep sessions. Processor 102 is capable of combining selected sleep sessions of the plurality of sleep sessions occurring in a same sleep cycle based on a maximum amount of time between the selected sleep sessions and the amounts of valid SpO₂ data collected for the selected sleep sessions.

For example, sleep session manager 216 is capable of determining that an insufficient amount of valid SpO₂ data was collected during a first sleep session of the plurality of sleep sessions of a selected sleep cycle and determining that a second sleep session of the plurality of sleep sessions occurs within a threshold amount of time after the end of the first sleep session in the selected sleep cycle. In response to determining that a sufficient amount of SpO₂ data is collected for the second sleep session, sleep session manager 216 determines the eAHI for the selected sleep cycle based on the SpO₂ data collected for the second sleep session (e.g., only using the SpO₂ data for the second sleep session while discarding the SpO₂ data from the first sleep session).

In one or more example implementations, sleep session manager 216 is capable of varying the maximum amount of time between the selected sleep sessions when determining whether to merge the selected sleep sessions based on the amounts of valid SpO₂ data collected for a first sleep session of a pair of sleep sessions considered for merging. Sleep session manager 216, for example, is capable of increasing the maximum amount of time between the selected sleep sessions in response to determining that the amount of valid SpO₂ data collected for the first sleep session is insufficient. An example of the merger process is described herein in greater detail in connection with FIG. 4.

In block 308, processor 102 in executing AHI estimator 220, estimates an eAHI for one or more of the plurality of sleep sessions including the merged sleep session.

In one or more examples, in the case where the merged sleep session is formed of a first sleep session and a second sleep session, the AHI estimator 220 estimates the eAHI for the merged sleep session by determining a first eAHI associated with the first sleep session based on a number of apneas during the first sleep session and determining a second eAHI associated with the second sleep session based on a number of apneas during the second sleep session. The AHI estimator 220 is capable of generating an eAHI for the merged sleep session by combining the first eAHI and the second eAHI.

In one or more examples, AHI estimator 220 generates the eAHI for the merged sleep session by combining the first eAHI and the second eAHI using a weighted sum. The weighted sum is determined using weights. The weights are determined based on a duration of the first sleep session and a duration of the second sleep session. For example, if the duration (3 hours) of the first sleep session is summed with the duration (1 hour) of the second sleep session, the weight of the each eAHI may be the percentage contribution of that sleep session to the duration of the merged sleep session. The weight of the eAHI of the first sleep session would be 0.75 while the weight of the eAHI of the second sleep session would be 0.25.

In block 310, processor 102 in executing sleep apnea detector 224, generates an indication of sleep apnea 226 specifying whether the user has sleep apnea based on one or more of the eAHIs 222 for one or more of the plurality of sleep cycles.

In one or more examples, sleep apnea detector 224, for each of the one or more sleep cycles, compares the eAHI associated with the sleep cycle with a threshold eAHI. Sleep apnea detector 224 is capable of indicating that the user has sleep apnea in response to determining that the eAHI associated with at least one sleep cycle exceeds the threshold eAHI.

FIG. 4 illustrates an example method 400 of generating and merging sleep sessions in accordance with the inventive arrangements disclosed herein. Method 400 is an example implementation of aspects of blocks 302, 304, 306, and 308 of FIG. 3 as performed by device 100 in executing SADS 110.

Method 400 may begin in block 402 where device 100 detects sleep onset. Sleep onset, e.g., the start of sleep for the user, may be detected by sleep session detector 208 using any of the various techniques described. In block 404, device 100 detects sleep offset, e.g., an arousal of the user. Sleep offset, e.g., an end of sleep for the user, may be detected by sleep session detector 208 as described.

In block 406, device 100 determines whether there is sufficient sleep data with valid SpO₂ data. In one aspect, device 100 determines whether a sum of the durations of all sleep sessions of the current sleep cycle, as defined by the respective sleep onset time and the sleep offset time of each respective sleep session, is at least 4 hours in duration. For example, sleep session detector 208 may indicate the duration of the sleep session. In addition, as discussed, SpO₂ evaluator 212 determines whether the sleep sessions for the current sleep cycle, taken cumulatively, have valid SpO₂ data. In response to determining that the total duration of the sleep sessions of the current sleep cycle is less than 4 hours or is at least 4 hours (4 hours or more) but has invalid SpO₂ data, method 400 continues to block 408. In response to determining that the total duration of the sleep sessions for the current sleep cycle is at least 4 hours and has valid SpO₂ data, method 400 continues to block 418.

For example, if a sleep session does not have a minimum number of valid SpO₂ values, device 100 considers the sleep session to be invalid and the SpO₂ data for that sleep session is not used. A preselected search window (e.g., in block 408) for a next sleep session in the same sleep cycle is initiated. If a subsequent sleep session among the valid sleep sessions occurs within the search window from the initial or prior sleep session, device 100 uses an eAHI from the each of the merged sleep sessions to determine the eAHI for the overall sleep cycle. The preselected search window may be shortened (e.g., in block 418) when sufficient sleep data are collected (e.g., at least 4 hours). In the example of FIG. 4, device 100 requires a minimum total sleep time of 4 hours in duration as measured across all merged sleep sessions for a sleep cycle to be valid for final classification. Use of the variable, maximum period of time ensures that if the arousal of the user is brief enough, device 100 considers the user to still be undergoing the same sleep cycle such that the two sleep sessions will merged.

Continuing with block 408, device 100 monitors for sleep onset occurring within a first maximum period of time. In one aspect, the first maximum period of time may be set to 90 minutes. In block 410, the device determines whether sleep onset was detected within the first maximum period of time. In response to detecting sleep onset within the first maximum period of time, method 400 loops back to block 402 to process the next detected sleep session as part of the same or current sleep cycle. In response to determining that no sleep onset was detected within the first maximum period of time, method 400 continues to block 412. In block 412, the device determines that there is insufficient sleep data for the user in order to estimate eAHI for the current sleep cycle. After block 412, method 400 continues to block 414.

In block 414, device 100 waits for a minimum amount of time before beginning to monitor for further sleep sessions. As an illustrative and non-limiting example, device 100 may wait for a period of 8 hours before attempting to detect sleep sessions again. In one aspect, the minimum amount of time to wait before attempting to detect sleep sessions is one that puts the start of sleep session detection into a next anticipated sleep cycle. The minimum amount of time may be referred to as a sleep refractory period during which the user is considered insusceptible to sleep or unlikely to sleep. During the sleep refractory period, device 100 operates on the premise that the user is unlikely to sleep long enough for device 100 to accurately evaluate sleep apnea.

In block 416, device 100 determines whether the minimum time has elapsed. In response to the minimum amount of time elapsing, method 400 loops back to block 402. In looping back to block 402 after block 416, device 100 considers a new sleep cycle to have begun. In response to the minimum amount of time not having elapsed, method 400 may loop back to block 414 to continue awaiting the expiration of the minimum amount of time.

Continuing with block 418 in the case where sufficient sleep data has been obtained that has valid SpO₂ data (e.g., a total duration of 4 or more hours of sleep sessions with valid SpO₂ data), device 100 monitors for sleep onset occurring within a second maximum period of time. In one aspect, the second maximum period of time may be set to 30 minutes. In block 420, device 100 determines whether sleep onset was detected within the second maximum period of time. In response to detecting sleep onset within the second maximum period of time, method 400 loops back to block 402 to process the next detected sleep session as part of the same or current sleep cycle. In response to determining that no sleep onset was detected within the second maximum period of time, method 400 continues to block 422. In block 422, the device considers the current sleep cycle to have ended and merges the sleep sessions that have valid SpO₂ data for the current sleep cycle.

In one aspect, as part of block 422, the device merges each sleep session of 1 hour or more in duration and that has valid SpO₂ data for the current sleep cycle. Other sleep sessions of less than an hour in duration and/or that do not have valid SpO₂ data are discarded. As part of the merging process performed in block 422, AHI estimator 220 is capable of calculating the eAHI for the sleep sessions, as merged. A weighted sum may be used to calculate the eAHI for the resulting merged sleep session. In block 425, device 100 optionally provides a notification to the user. The notification, which may be provided via a user interface and/or graphical user interface of the device may indicate that a classification result was obtained for the current sleep cycle. The indication may also specify that sleep monitoring for approximately ½ of the sleep cycle was completed. After block 422, method 400 continues to block 414.

As discussed, when merging eAHI values, the eAHI values are combined using a weighted sum technique. When multiple sleep cycles of data are collected, the eAHI of each sleep cycle may be compared against a clinical threshold (e.g., 15 AHI) and counted as a positive or a negative vote for sleep apnea based on whether the threshold was exceeded. The threshold of 15 is used since 15 on the AHI scale is used as the demarcation of moderate to severe sleep apnea. In determining whether a user has sleep apnea, sleep apnea detector 224 may be configured to employ any of a variety of different evaluation techniques. For example, sleep apnea detector 224 is capable of being configured to check whether any, all, or a minimum percentage of votes from each sleep cycle of a plurality of sleep cycles is/are positive. In one or more particular examples, device 100 may be configured to use two sleep cycles of positive votes that occur within a predetermined number of sleep cycles such as 10. This latter technique may be used to achieve high levels of accuracy that reduces false positives in the context of an OTC ambulatory care.

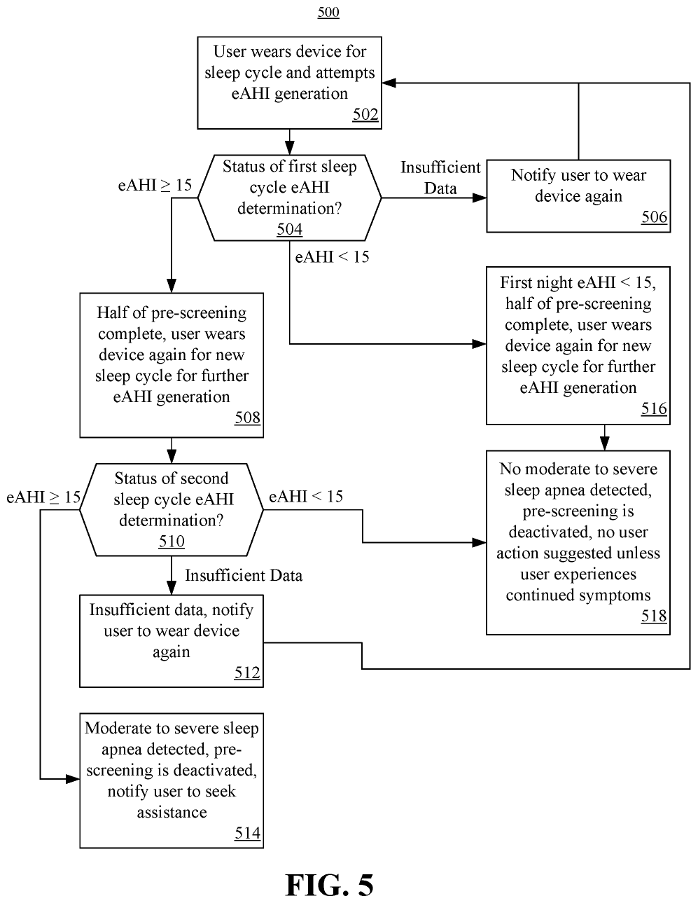

FIG. 5 illustrates an example method 500, as performed by device 100 in executing SADS 110, of determining whether the user has experienced moderate to severe sleep apnea. The example of FIG. 5 illustrates a scenario in which device 100 monitors the sleep of the user over a plurality of sleep cycles. In order to determine that a user has moderate to severe sleep apnea, device 100 must detect a measure of sleep apnea above a threshold for at least two nights. In this example, the two nights must be within 10 days of one another or otherwise are considered unrelated.

In block 502, the user wears device 100 for a sleep cycle and device 100 attempts to generate an eAHI for that sleep cycle. In block 504, device 100 evaluates the result from block 502. In general, the eAHI for each sleep cycle is compared against a clinical sleep apnea AHI threshold of 15 to determine whether the user experienced significant sleep apnea during that sleep cycle.

In response to determining that the eAHI is greater than or equal to 15, method 500 continues to block 508. In response to determining that the eAHI is less than 15, method 500 continues to block 516. In response to determining that there was insufficient data to determine an eAHI for the current sleep cycle, method 500 continues to block 506. In block 506, since insufficient data was collected, device 100 notifies the user to wear the device again for a next and/or different sleep cycle.

In block 508, where the eAHI was determined to be greater than or equal to 15, method 500 determines that half of the screening has completed. Device 100 directs the user to wear to the device again for a next and/or different sleep cycle. Accordingly, the user wears the device again for a next and/or different sleep cycle. In one or more example implementations, the next sleep cycle must be within a predetermined amount of time, e.g., number of sleep cycles, of the first sleep cycle having an eAHI of 15 or more. The predetermined amount of time may be 10 days or 10 sleep cycles. That is, if the first sleep cycle (e.g., first night) has an eAHI of 15 or more, the next sleep cycle with an eAHI of 15 or more leading to the conclusion that the user suffers from moderate to several sleep apnea must occur on or before the 10^{th}sleep cycle (e.g., 10^{th} night). The example of FIG. 5 illustrates an embodiment where device 100 only provides a final classification based on multiple sleep cycles of valid sleep data. The number of sleep cycles used for classification can be customized based on the use case. For example, a clinical setting may use 2 nights, while at home general use setting may be 2-4 nights.

In block 510, device 100 evaluates the result from block 508. In response to determining that the eAHI is greater than or equal to 15, method 500 continues to block 514. In block 514, the device indicates that the user has experienced moderate to severe sleep apnea. In block 514, sleep apnea pre-screening is deactivated and the user is notified to seek assistance and/or to consult a sleep specialist. In response to determining that the eAHI is less than 15, method 500 continues to block 518. In block 518, the device determines that moderate to severe sleep apnea was not detected. Prescreening is deactivated. No user action is suggested. In one or more examples, user action may be suggested if the user experiences continued symptoms.

If the result of block 508 is that there was insufficient data to determine an eAHI for the second sleep cycle, method 500 continues to block 512. In block 512, the device notifies the user to wear the device again to monitor sleep for a next sleep cycle. After block 512, method 500 may loop back to block 502.

The example of FIG. 5 illustrates a particular implementation of sleep apnea detection. In other examples, the configuration of sleep apnea detector 224 may be adjusted or customized based on use case. In general use, for example, sleep apnea detector 224 may require two sleep cycles with eAHIs exceeding 15 to detect sleep apnea. In other examples, sleep apnea detector 224 may use a different number of sleep cycles, e.g., only 1 or more than 2, to detect sleep apnea. In addition, the time limit for detecting the predetermined and configurable number of eAHIs exceeding 15 may be adjusted to a number less than 10, 10, or more than 10.

In one or more example implementations, the operations described within this disclosure may be performed entirely using device 100. In one or more other example implementations, where additional processing power is required, some operations may be offloaded to another device that is coupled (e.g., communicatively linked) with device 100. For example, operations such as adapting or adjusting one or more of the models over time and/or performing a self-learning operation may be offloaded to a coupled device such as a smart phone or other computing system and provide the updated model(s) back to device 100.

The terminology used herein is for the purpose of describing particular embodiments only and is not intended to be limiting. Notwithstanding, several definitions that apply throughout this document now will be presented.

As defined herein, the singular forms “a,” “an,” and “the” are intended to include the plural forms as well, unless the context clearly indicates otherwise.

The term “approximately” means nearly correct or exact, close in value or amount but not precise. For example, the term “approximately” may mean that the recited characteristic, parameter, or value is within a predetermined amount of the exact characteristic, parameter, or value.

As defined herein, the terms “at least one,” “one or more,” and “and/or,” are open-ended expressions that are both conjunctive and disjunctive in operation unless explicitly stated otherwise. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, the term “automatically” means without user intervention.

As defined herein, the term “computer readable storage medium” means a storage medium that contains or stores program code for use by or in connection with an instruction execution system, apparatus, or device. As defined herein, a “computer readable storage medium” is not a transitory, propagating signal per se. A computer readable storage medium may be, but is not limited to, an electronic storage device, a magnetic storage device, an optical storage device, an electromagnetic storage device, a semiconductor storage device, or any suitable combination of the foregoing. The different types of memory, as described herein, are examples of a computer readable storage media. A non-exhaustive list of more specific examples of a computer readable storage medium may include: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, or the like.

As defined herein, the term “if” means “when” or “upon” or “in response to” or “responsive to,” depending upon the context. Thus, the phrase “if it is determined” or “if [a stated condition or event] is detected” may be construed to mean “upon determining” or “in response to determining” or “upon detecting [the stated condition or event]” or “in response to detecting [the stated condition or event]” or “responsive to detecting [the stated condition or event]” depending on the context.

As defined herein, the terms “one embodiment,” “an embodiment,” “one or more embodiments,” or similar language mean that a particular feature, structure, or characteristic described in connection with the embodiment is included in at least one embodiment described within this disclosure. Thus, appearances of the phrases “in one embodiment,” “in an embodiment,” “in one or more embodiments,” and similar language throughout this disclosure may, but do not necessarily, all refer to the same embodiment. The terms “embodiment” and “arrangement” are used interchangeably within this disclosure.

As defined herein, the term “output” means storing in physical memory elements, e.g., devices, writing to a display or other peripheral output device, sending or transmitting to another system, exporting, or the like.

As defined herein, the term “processor” means at least one hardware circuit. The hardware circuit may be configured to carry out instructions contained in program code. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

As defined herein, the term “real-time” means a level of processing responsiveness that a user or system senses as sufficiently immediate for a particular process or determination to be made, or that enables the processor to keep up with some external process.

As defined herein, the term “responsive to” and similar language as described above, e.g., “if,” “when,” or “upon,” mean responding or reacting readily to an action or event. The response or reaction is performed automatically. Thus, if a second action is performed “responsive to” a first action, there is a causal relationship between an occurrence of the first action and an occurrence of the second action. The term “responsive to” indicates the causal relationship.

The term “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

As defined herein, the term “user” means a human being.

The terms first, second, etc. may be used herein to describe various elements. These elements should not be limited by these terms, as these terms are only used to distinguish one element from another unless stated otherwise or the context clearly indicates otherwise.

A computer program product may include a computer readable storage medium (or media) having computer readable program instructions thereon for causing a processor to carry out aspects of the present invention. Within this disclosure, the term “program code” is used interchangeably with the term “computer readable program instructions.” Computer readable program instructions described herein may be downloaded to respective computing/processing devices from a computer readable storage medium or to an external computer or external storage device via a network, for example, the Internet, a LAN, a WAN and/or a wireless network. The network may include copper transmission cables, optical transmission fibers, wireless transmission, routers, firewalls, switches, gateway computers and/or edge devices including edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations for the inventive arrangements described herein may be assembler instructions, instruction-set-architecture (ISA) instructions, machine instructions, machine dependent instructions, microcode, firmware instructions, or either source code or object code written in any combination of one or more programming languages, including an object-oriented programming language and/or procedural programming languages. Computer readable program instructions may specify state-setting data. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some cases, electronic circuitry including, for example, programmable logic circuitry, an FPGA, or a PLA may execute the computer readable program instructions by utilizing state information of the computer readable program instructions to personalize the electronic circuitry, in order to perform aspects of the inventive arrangements described herein.

Certain aspects of the inventive arrangements are described herein with reference to flowchart illustrations and/or block diagrams of methods, apparatus (systems), and computer program products. It will be understood that each block of the flowchart illustrations and/or block diagrams, and combinations of blocks in the flowchart illustrations and/or block diagrams, may be implemented by computer readable program instructions, e.g., program code.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In this way, operatively coupling the processor to program code instructions transforms the machine of the processor into a special-purpose machine for carrying out the instructions of the program code. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the operations specified in the flowchart and/or block diagram block or blocks.

The computer readable program instructions may also be loaded onto a computer, other programmable data processing apparatus, or other device to cause a series of operations to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the inventive arrangements. In this regard, each block in the flowcharts or block diagrams may represent a module, segment, or portion of instructions, which comprises one or more executable instructions for implementing the specified operations. In some alternative implementations, the operations noted in the blocks may occur out of the order noted in the figures. For example, two blocks shown in succession may be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts or carry out combinations of special purpose hardware and computer instructions.

The corresponding structures, materials, acts, and equivalents of all means or step plus function elements that may be found in the claims below are intended to include any structure, material, or act for performing the function in combination with other claimed elements as specifically claimed.

The description of the embodiments provided herein is for purposes of illustration and is not intended to be exhaustive or limited to the form and examples disclosed. The terminology used herein was chosen to explain the principles of the inventive arrangements, the practical application or technical improvement over technologies found in the marketplace, and/or to enable others of ordinary skill in the art to understand the embodiments disclosed herein. Modifications and variations may be apparent to those of ordinary skill in the art without departing from the scope and spirit of the described inventive arrangements. Accordingly, reference should be made to the following claims, rather than to the foregoing disclosure, as indicating the scope of such features and implementations.

What is claimed is:

1. A method within and by a wearable device including a hardware processor, the method comprising:
while a user is wearing the wearable device during one or more sleep cycles, continuously monitoring, by the hardware processor, sensor signals generated by sensors of the wearable device to detect a plurality of sleep sessions occurring during the one or more sleep cycles;
measuring, by the hardware processor and based on the sensor signals, amounts of valid SpO₂ data collected for the plurality of sleep sessions;
generating, by the hardware processor, a merged sleep session by combining selected sleep sessions of the plurality of sleep sessions occurring in a same sleep cycle based on a maximum amount of time between the selected sleep sessions and the amounts of valid SpO₂ data collected for the selected sleep sessions;
estimating, by the hardware processor, an estimated apnea hypopnea index (eAHI) for the one or more sleep sessions including the merged sleep session; and
generating, by the hardware processor, an indication of whether the user has sleep apnea based on one or more of the eAHIs for the one or more sleep cycles.

2. The method of claim 1, wherein the generating the indication comprises:
for each of the one or more sleep cycles, comparing the eAHI associated with the sleep cycle with a threshold eAHI; and
indicating that the user has sleep apnea in response to determining that the eAHI associated with at least one sleep cycle exceeds the threshold eAHI.

3. The method of claim 1, wherein the generating the merged sleep session comprises:
determining that an insufficient amount of valid SpO₂ data was collected during a first sleep session of the plurality of sleep sessions of a selected sleep cycle;
determining that a second sleep session of the plurality of sleep sessions occurs within a threshold amount of time after an end of the first sleep session in the selected sleep cycle; and
in response to determining that a sufficient amount of SpO₂ data is collected for the second sleep session, determining the eAHI associated with the selected sleep cycle based on the SpO₂ data collected for the second sleep session.

4. The method of claim 1, wherein the merged sleep session is formed of a first sleep session and a second sleep session, and wherein the estimating the eAHI comprises, for the merged sleep session:
determining a first eAHI associated with the first sleep session based on a number of apneas during the first sleep session;
determining a second eAHI associated with the second sleep session based on a number of apneas during the second sleep session; and
generating an eAHI for the merged sleep session by combining the first eAHI and the second eAHI.

5. The method of claim 4, further comprising:
combining the first eAHI and the second eAHI using a weighted sum; and
determining the weighted sum using weights determined based on a duration of the first sleep session and a duration of the second sleep session.

6. The method of claim 1, further comprising:
varying the maximum amount of time between the selected sleep sessions when determining whether to merge the selected sleep sessions based on the amounts of valid SpO₂ data collected for a first sleep session of the selected sleep sessions considered for merging.

7. The method of claim 6, wherein the maximum amount of time between the selected sleep sessions is increased in response to determining that the amount of valid SpO₂ data collected for the first sleep session is insufficient.

8. A system, comprising:
a plurality of sensors configured to generate sensor signals; and
a hardware processor coupled to the plurality of sensors, the hardware processor configured to continuously monitor the sensor signals while worn by a user during one or more sleep cycles, wherein the hardware processor includes:
a sleep session detector configured to detect a plurality of sleep sessions occurring during the one or more sleep cycles based on the sensor signals;
an SpO₂ quantifier configured to determine amounts of SpO₂ data collected for the plurality of sleep sessions based on the sensor signals;
a sleep session manager configured to generate a merged sleep session by combining selected sleep sessions of the plurality of sleep sessions occurring in a same sleep cycle based on a maximum amount of time between the selected sleep sessions and the amounts of SpO₂ data collected for the selected sleep sessions;
an apnea hypopnea index (AHI) estimator configured to estimate an estimated apnea hypopnea index (eAHI) for one or more of the plurality of sleep sessions including the merged sleep session; and
a sleep apnea detector configured to indicate, via a user interface of the system, whether the user has sleep apnea based on one or more eAHIs for the one or more sleep cycles.

9. The system of claim 8, wherein the sleep apnea detector is configured to:
for each of the one or more sleep cycles, compare the eAHI associated with the sleep cycle with a threshold eAHI; and
indicate that the user has sleep apnea in response to determining that the eAHI associated with at least one sleep cycle exceeds the threshold eAHI.

10. The system of claim 8, wherein the sleep session manager is configured to:
determine that an insufficient amount of valid SpO₂ data was collected during a first sleep session of the plurality of sleep sessions of a selected sleep cycle;
determine that a second sleep session of the plurality of sleep sessions occurs within a threshold amount of time after an end of the first sleep session in the selected sleep cycle; and
in response to determining that a sufficient amount of SpO₂ data is collected for the second sleep session, determine the eAHI associated with the selected sleep cycle based on the SpO₂ data collected for the second sleep session.

11. The system of claim 8, wherein the merged sleep session is formed of a first sleep session and a second sleep session, and wherein, for the merged sleep session, the AHI estimator is configure to:
determine a first eAHI associated with the first sleep session based on a number of apneas during the first sleep session;
determine a second eAHI associated with the second sleep session based on a number of apneas during the second sleep session; and
generate an eAHI for the merged sleep session by combining the first eAHI and the second eAHI.

12. The system of claim 11, wherein AHI estimator is configured to:
combine the first eAHI and the second eAHI using a weighted sum; and
determine the weighted sum using weights determined based on a duration of the first sleep session and a duration of the second sleep session.

13. The system of claim 8, wherein the sleep session manager is configured to:
vary the maximum amount of time between the selected sleep sessions when determining whether to merge the selected sleep sessions based on the amounts of valid SpO₂ data collected for a first sleep session of the selected sleep sessions considered for merging.

14. The system of claim 13, wherein the maximum amount of time between the selected sleep sessions is increased in response to determining that the amount of valid SpO₂ data collected for the first sleep session is insufficient.

15. A computer program product, comprising:
one or more computer readable storage media, and program instructions collectively stored on the one or more computer readable storage media, wherein the program instructions are executable by computer hardware of a device to initiate operations including:
while a user is wearing the device during one or more sleep cycles, continuously monitoring sensor signals generated by sensors of the device to detect a plurality of sleep sessions occurring during the one or more sleep cycles;
measuring, based on the sensor signals, amounts of valid SpO₂ data collected for the plurality of sleep sessions;
generating a merged sleep session by combining selected sleep sessions of the plurality of sleep sessions occurring in a same sleep cycle based on a maximum amount of time between the selected sleep sessions and the amounts of valid SpO₂ data collected for the selected sleep sessions;
estimating an estimated apnea hypopnea index (eAHI) for one or more of the plurality of sleep sessions including the merged sleep session; and
generating an indication of whether the user has sleep apnea based on one or more of the eAHIs for the one or more sleep cycles.

16. The computer program product of claim 15, wherein the generating the merged sleep session comprises:
determining that an insufficient amount of valid SpO₂ data was collected during a first sleep session of the plurality of sleep sessions of a selected sleep cycle;
determining that a second sleep session of the plurality of sleep sessions occurs within a threshold amount of time after an end of the first sleep session in the selected sleep cycle; and
in response to determining that a sufficient amount of SpO₂ data is collected for the second sleep session, determining the eAHI associated with the selected sleep cycle based on the SpO₂ data collected for the second sleep session.

17. The computer program product of claim 15, wherein the merged sleep session is formed of a first sleep session and a second sleep session, and wherein the estimating the eAHI comprises, for the merged sleep session:
determining a first eAHI associated with the first sleep session based on a number of apneas during the first sleep session;
determining a second eAHI associated with the second sleep session based on a number of apneas during the second sleep session; and
generating an eAHI for the merged sleep session by combining the first eAHI and the second eAHI.

18. The computer program product of claim 17, wherein the program instructions are executable by the computer hardware to initiate operations further comprising:
combining the first eAHI and the second eAHI using a weighted sum; and
determining the weighted sum using weights determined based on a duration of the first sleep session and a duration of the second sleep session.

19. The computer program product of claim 15, wherein the program instructions are executable by the computer hardware to initiate operations further comprising:
varying the maximum amount of time between the selected sleep sessions when determining whether to merge the selected sleep sessions based on the amounts of valid SpO₂ data collected for a first sleep session of the selected sleep sessions considered for merging.

20. The computer program product of claim 19, wherein the maximum amount of time between the selected sleep sessions is increased in response to determining that the amount of valid SpO₂ data collected for the first sleep session is insufficient.
